(19) United States
(12) Patent Application Publication
(10) Pub. No.: US 2009/0093446 A1
(43) Pub. Date: Apr. 9, 2009

(54) METHOD FOR ALLEVIATING KERATOCONJUNCTIVITIS SICCA

(75) Inventor: Joel E. Bernstein, Deerfield, IL (US)

Correspondence Address: BARNES & THORNBURG LLP, P.O. BOX 2786, CHICAGO, IL, 60690-2786, US

(73) Assignee: WINSTON LABORATORIES, INC., Vernon Hills, IL (US)

(21) Appl. No.: 11/868,286

(22) Filed: Oct. 5, 2007

(51) Int. Cl. A61K 31/165; A61K 31/167; A61K 31/565; A61K 31/57

(52) U.S. Cl. 514/171; 514/627; 514/626

(57) ABSTRACT

An improved method of increasing tear production is by intranasally administering a therapeutically effective amount of a capsaicinoid compound to patients with deficient tear production. Optional incorporation into the intranasal formulation of a topical corticosteroid or topical anesthetic compound is used to reduce transient nasal stinging and burning which may sometimes accompany intranasal administration of capsaicinoids.

### BACKGROUND

As many as 9 million Americans over 50 years of age suffer from a condition called keratoconjunctivitis sicca or dry eye. Discomfort from dry eye ranges from a mild burning to a persistent sense of scratching under the lids. Dry eye is not just painful, but the condition can predispose to eye infections and also produce blurred vision. Dry eye is thought to be due to either an inability to produce sufficient tears or inflammation in the external eye.

Previous and current attempts to treat dry eye have included various artificial tear drops as well as ophthalmic drops containing cyclosporine, a drug to reduce inflammation in the eye. Both artificial tear drops and cyclosporine drops frequently cause local irritation with burning and stinging, can blur vision and are poorly effective. In clinical studies of patients treated with cyclosporine drops, only 15% of patients treated with cyclosporine drops demonstrated any increase in tear production over a 6-month treatment period. There is consequently a substantial need for therapies for dry eye which are more effective and which may produce less in the way of adverse ocular reactions such as stinging or blurry vision. The present disclosure provides such methods and compositions.

### SUMMARY

An improved method of increasing tear production is by intranasally administering a therapeutically effective amount of a capsaicinoid compound to patients with deficient tear production. It was surprising and unexpected to use a nasal route of administration to treat an ocular condition. To the inventor's knowledge, no nasal preparations have been reported for use to treat eyes. Optional incorporation into the intranasal formulation of a topical corticosteroid or topical anesthetic compound is used to reduce transient nasal stinging and burning which may sometimes accompany intranasal administration of capsaicinoids.

A method of treating keratoconjunctivitis sicca, also known as dry eye is, by administration of an effective amount of a composition suitable for intranasal administration containing capsaicinoid compounds to the nasal mucosa with a resulting significant increase in production of ocular tearing and a reduction in burning, stinging, blurring of vision and other adverse symptoms and signs of dry eye.

A suitable capsaicinoid compound includes capsaicin, civamide, acetylated congenures of capsaicin and civamide, or salts of all of the aforementioned capsaicinoids.

The capsaicinoid compound is present within the formulation in a range of between about 0.001% by weight to about 5.0% by weight.

A vehicle for a composition suitable for administration to the nasal mucosa is a solution, suspension, cream, ointment, gel or mucosal patches.

A topical anesthetic or a topical corticosteroid is optionally included with the capsaicinoid in order to reduce irritation of the nasal mucosa which may be produced by capsaicinoids. A suitable topical anesthetic is pramoxine, lidocaine, dibucaine, prilocaine, their salts and related compounds. The topical anesthetic is present in the amount of about 0.1% to about 5.0% by weight. A suitable topical corticosteroid includes hydrocortisone, triamcinolone, betamethasone, their salts and related compounds. These topical corticosteroids are present in the amount of about 0.01% to about 2.5% by weight.

### DETAILED DESCRIPTION

The inventor has recently discovered a novel method of producing increased tear production (lacrimation) without the adverse intraocular side effects noted with the variety of eye drops utilized to treat patients with dry eye. This method consists of the intranasal administration of a class of chemicals called capsaicinoids, resulting in increased tear production without significant irritation to the eyes themselves. The inventor discovered that administration of capsaicinoid compounds for migraine relief resulted in a surprising finding of increased tearing in individuals.

Among the safest and most effective capsaicinoid utilized in this new method is civamide (cis-8-methyl-N-vanillyle-nonenamide) a chemical which has been the subject of two previous U.S. Patents (U.S. Pat. Nos. 5,063,060 and 7,244,446) incorporated here by reference. In the course of conducting a number of clinical investigations utilizing intranasal capsaicinoids for treatment of headache and neuralgia pain, as well as nasal stuffiness, it was discovered surprisingly that intranasally administered capsaicinoids produce increased ocular tear production without frequent locally adverse effects on the eyes.

In accordance with the invention, formulations suitable for intranasal administration, such as solutions, suspensions, lotions, creams and gels, containing about 0.001% to about 5.0% by weight of capsaicin, civamide, or acetylated derivatives of capsaicin and civamide, are introduced into the nasal passages by a drop or spray of a solution or suspension of the capsaicinoid compound, as well as application to the nasal mucosa in the form of a gel, cream, lotion or ointment.

While the above described method produces effective stimulus of increased tear production without untoward ocular side effects such as stinging or blurred vision in the eyes, many individuals will experience transient burning or stinging sensations in their nasal mucosa. The compositions described are modified to reduce the frequency and/or severity of these intranasal side effects by incorporating into the capsaicinoid compositions for nasal administration amounts of either a local anesthetic (0.1 to 5% by weight) or a topically effective corticosteroid (0.01 to 2.5% by weight). Such local anesthetics include, for example, lidocaine, prilocaine, pramoxine, and dibucaine. Such topically effective corticosteroids include, for example, hydrocortisone and its various salts, triamcinolone and its salts, and betamethasone and its salts.

A method of quantitatively determining the amount of tearing is by weighing a dry filter or absorbent paper before applying the tears and measuring the weight after application of tears. The difference in weight provides the amount of tears secreted.

### EXAMPLES

Examples are provided for illustrative purposes and are not intended to limit the scope of the disclosure.

### Example 1

Thirty (30) normal volunteers divided into 3 separate 10 subject panels participated in a multi-dose 14 day tolerance study evaluating the local effects of 0.0075% by weight civamide nasal spray administered twice daily, 0.01% by weight civamide administered twice daily, and 0.015% by weight civamide administered once daily. Ten (10) of 10 (100%) of the subjects in each of the two groups receiving civamide twice daily related that they experienced increased lacrimation (tear production), while 9 of 10 (90%) of subjects utilizing 0.015% by weight civamide once daily experienced increased tearing.

### Example 2

Fifty-five (55) patients with vasomotor rhinitis (non-allergic rhinitis) participated in a double-blind placebo-controlled 2 week evaluation of the symptomatic relief afforded patients by 0.01% by weight civamide nasal spray or placebo nasal spray (vehicle for active product). Thirty-one percent (31%) of patients using 0.01% civamide spray reported increased tearing, while 0% of patients on placebo noted such a side effect.

### Example 3

Thirty-four (34) patients with migraine headache applied 0.025% by weight capsaicin cream to their nasal mucosa for relief of their headache. Seventy-three percent (73%) of such patients noted some relief of their headaches 4 hours after administration of 0.025% capsaicin cream. Forty-four percent (44%) of these patients reported increased tearing as a side effect.

### Example 4

In a study utilizing 0.025% by weight civamide nasal drops administered once daily for 1 week to 28 patients with episodic cluster headache (a severe form of vasomotor headache that occurs in general 4-24 week clusters), 9 of 18 patients (50%) on civamide nasal drops had increased tearing versus 0 of 10 patients (0%) on vehicle control.

### Example 5

In 112 episodic cluster headache patients administered either 0.01% by weight civamide nasal spray or inactive control (10% NaCl) twice daily, 36 patients (51%) on civamide spray and 3 patients (7%) on control reported increased tearing.

### Example 6

Two patients with migraine headache disorder were administered 0.025% by weight capsaicin cream intranasally for acute relief of their migraine pain. Both reported increased lacrimation within 30 minutes of application of the capsaicin cream.

### Example 7

Formulations of civamide nasal spray can be prepared with low doses of various local (i.e. topical) anesthetics from 0.1% to 5.0% by weight of pramoxine, lidocaine, dibucaine, prilocaine, their salts and related compounds, or topical corticosteroids from 0.01% to 2.5% by weight, including hydrocortisone, triamcinolone, betamethasone, their salts and related compounds to reduce initial nasal irritant reactions produced by capsaicinoid compounds.

While the foregoing is a description of the preferred embodiments of the invention, it will be readily apparent to those skilled in the art that various modifications may be made therein without departing from the true scope and spirit of the invention as set forth in the appended claims.

1. A method of treating keratoconjunctivitis sicca by administration of an effective amount of a composition comprising capsaicinoid compounds, suitable for intranasal administration to the nasal mucosa.

2. The method of claim 1, wherein said capsaicinoid compound is selected from the group consisting of capsaicin, civamide, acetylated congenures of capsaicin and civamide, and salts of all of the aforementioned capsaicinoids.

3. The method of claim 2, wherein the capsaicinoid compound is present within the composition in a range of between about 0.001% by weight to about 5.0% by weight.

4. The method of claim 1, further comprising a vehicle for a composition suitable for administration to the nasal mucosa, wherein the vehicle is selected from the group consisting of solutions, suspensions, creams, ointments, gels and mucosal patches.

5. The method of claim 2, further comprising a topical anesthetic or a topical corticosteroid.

6. The method of claim 5, in which said topical anesthetic is selected from a group which includes pramoxine, lidocaine, dibucaine, prilocaine, their salts and compounds of essentially the same function.

7. The method of claim 6, wherein said topical anesthetic is present in the amount of about 0.1% to about 5.0% by weight.

8. The method of claim 5, wherein said topical corticosteroid is selected from a group which includes hydrocortisone, triamcinolone, betamethasone, their salts and related compounds.

9. The method of claim 8, wherein the topical corticosteroid is present in the amount of about 0.01% to about 2.5% by weight.
